(19) United States
(12) Patent Application Publication
(10) Pub. No.: US 2023/0404877 A1
(43) Pub. Date: Dec. 21, 2023

(54) POWDER-CONTAINING COMPOSITION

(71) Applicant: Shiseido Company, Ltd., Tokyo (JP)

(72) Inventors: Toru KINA, Tokyo (JP); Yuuji SONOYAMA, Tokyo (JP); Mitsuki NAKAMURA, Tokyo (JP)

(73) Assignee: Shiseido Company, Ltd., Tokyo, (JP)

(21) Appl. No.: 18/028,690

(22) PCT Filed: Oct. 19, 2021
(86) PCT No.: PCT/JP2021/038603

§ 371 Date: Mar. 27, 2023

(30) Foreign Application Priority Data
Oct. 26, 2020 (JP) 2020-178825
Oct. 26, 2020 (JP) 2020-178826
May 21, 2021 (JP) 2021-086457

(51) Int. Cl. A61K 8/34; A61P 19/00; A61K 8/02; A61K 8/86; A61K 8/25

(52) U.S. Cl. CPC A61K 8/345; A61P 19/00; A61K 8/025; A61K 2800/622; A61K 8/86; A61K 8/25; A61K 2800/10; A61K 8/0275

(57) ABSTRACT

A powder-containing composition contains from 0.5% by mass to 22% by mass of a first powder which is a nonspherical powder having a hydrophobic particle surface, from 6% by mass to 25% by mass of a liquid-state polyol, 50% by mass or greater of water, and from 3% by mass to 12% by mass of a liquid-state oily component. A content of a second powder which is a spherical powder is 1.5 parts by mass or less relative to 1 part by mass of the first powder. A content of a surfactant is 0.5% by mass or less relative to the mass of the powder-containing composition.

### CROSS REFERENCE TO RELATED APPLICATIONS

The present application is based upon and claims the benefit of the priorities of Japanese Patent Application No. 2020-178825 (filed on Oct. 26, 2020), Japanese Patent Application No. 2020-178826 (filed on Oct. 26, 2020), and Japanese Patent Application No. 2021-86457 (filed on May 21, 2021), the disclosures of which are incorporated herein in its entirety by reference.

### TECHNICAL FIELD

The present disclosure relates to a composition containing powder.

### BACKGROUND ART

Cosmetic compositions containing powder are known in the art. For example, Patent Literature 1 discloses thinly-applicable cosmetic preparations having a creamy or paste-like consistency.

A cosmetic preparation disclosed in Patent Literature 1 contains about 80% by mass of water, 5% by mass of hydrophobized silicon dioxide powder, 2.5% by mass of hydrophilized mica, 5% by mass of pentylene glycol, 2% by mass of dimethicone, 0.5% by mass of dimethicone copolyol, 0.75% by mass of sodium carboxymethyl starch, etc.

### CITATION LIST

### Patent Literature

Patent Literature 1: PCT Japanese National Phase Publication No. 2007-512380

### SUMMARY OF INVENTION

### Technical Problem

The following analysis can be made from the perspective of the present disclosure.

In powder-containing compositions, powder needs to be dispersed within the composition. The cosmetic preparation disclosed in Patent Literature 1 maintains composition stability by adding hydrophobized silicon dioxide powder, which is a spherical powder. Unfortunately, containing an excessive amount of spherical particles (spherical powders) may lead to uneven application due to the spherical powders at the time of application, which may impair uniform luster. On the other hand, if the amount of liquid-state polyol is too small, the user may feel tautness at the time of application, which may intensify the squeaky-powdery feel.

Further, the cosmetic preparation disclosed in Patent Literature 1 does not have a sufficient oil content, and thus, the user cannot feel moistness even when the preparation is applied to the skin.

Hence, there has been a demand for a powder-containing composition that is capable of creating a uniform coating film while having stable powder dispersibility, and with which squeakiness is suppressed at the time of application and the user can feel moistness when the composition is applied to the skin.

### Solution to Problem

According to a first aspect of the present disclosure, a powder-containing composition is provided, the composition comprising from 0.5% by mass to 22% by 30 mass of a first powder which is a nonspherical powder having a hydrophobic particle surface, from 6% by mass to 25% by mass of a liquid-state polyol, 50% by mass or greater of water, and from 3% by mass to 12% by mass of a liquid-state oily component. A content of a second powder which is a spherical powder is 1.5 parts by mass or less relative to 1 part by mass of the first powder. A content of a surfactant is 0.5% by mass or less relative to the mass of the powder-containing composition.

### Advantageous Effects of Invention

With the powder-containing composition of the present disclosure, powder can be stably dispersed within the powder-containing composition while suppressing aggregation without requiring a surfactant.

With the powder-containing composition of the present disclosure, the powder contained in the composition can be applied uniformly to an application target (e.g., the skin). Thus, the appearance of the application region can be improved. Further, by enabling the powder to adhere uniformly to an application target (e.g., the skin), secondary adhesion to masks, clothing, etc., can be suppressed.

With the powder-containing composition of the present disclosure, squeakiness arising at the time of application can be suppressed.

With the powder-containing composition of the present disclosure, the user can feel moistness when applying the powder-containing composition to the skin.

### DESCRIPTION OF EMBODIMENTS

Preferred modes of the aforementioned aspects will be described below.

According to a preferred mode of the aforementioned first aspect, the first powder is at least one selected from the group consisting of platy powders and blocky powders.

According to a preferred mode of the aforementioned first aspect, the first powder has an aspect ratio of from 1.5 to 170.

According to a preferred mode of the aforementioned first aspect, the second powder has an average aspect ratio of less than 1.2.

According to a preferred mode of the aforementioned first aspect, the oily component is at least one selected from the group consisting of fluoro-oils, silicone oils, polar oils, and hydrocarbon oils.

According to a preferred mode of the aforementioned first aspect, the first powder is a platy powder coated by at least one selected from the group consisting of higher fatty acids, metallic soaps, silicone compounds, surfactants, and dextrin fatty acid esters.

According to a preferred mode of the aforementioned first aspect, the liquid-state polyol is at least one selected from the group consisting of glycerin, 1,3-butylene glycol, polyethylene glycol, diglycerin, and dipropylene glycol.

According to a preferred mode of the aforementioned first aspect, the powder-containing composition further comprises at least one selected from the group consisting of PEG-240/HDI copolymer bis-decyltetradeceth-20 ether, dimethylacrylamide/sodium acryloyldimethyltaurate crosspolymer, and polyurethane-59.

According to a preferred mode of the aforementioned first aspect, the powder-containing composition has a viscosity of from 1,000 mPa·s to 150,000 mPa·s. According to a preferred mode of the aforementioned first aspect, the liquid-state oily component and the water are a mixture in a state of a giant emulsion.

According to a preferred mode of the aforementioned first aspect, the powder-containing composition is for use in a cosmetic.

In the following description, POE is an abbreviation of polyoxyethylene, POP is an abbreviation of polyoxypropylene, and the number in parentheses after POE or POP indicates the average number of moles of POE groups or POP groups added in the compound concerned.

In the present disclosure, “substantial amount” refers to an amount capable of bringing about the functions/effects achieved by the addition of the compound concerned.

A powder-containing composition according to a first embodiment of the present disclosure will be described.

In the present disclosure, “liquid-state” refers to a form/state that has flowability and is a liquid at 25° C.

The powder-containing composition of the present disclosure contains a first powder, a liquid-state polyol, water, and a liquid-state oily component. It is preferred that the powder-containing composition of the present disclosure is a liquid-state composition under atmospheric pressure at 25° C.

First Powder:

The first powder is a powder having a hydrophobic particle surface. For example, the first powder may be a powder whose particle surface has been hydrophobized. An example of a hydrophobizing method may be to coat the particles with at least one selected from the group consisting of higher fatty acids, metallic soaps, silicone compounds, surfactants, and dextrin fatty acid esters. The particle surface of the first powder does not have to be hydrophobized so long as the particles are hydrophobic.

The first powder is nonspherical. For example, the first powder may be at least one selected from the group consisting of platy powders and blocky powders (aggregated powders). A platy powder is a powder whose primary particles have a plate shape. A blocky powder is a powder including secondary particles having an amorphous shape formed by aggregation of primary particles. In blocky powders, it is preferred that the primary particles have a hydrophobic particle surface.

It is preferred that the first powder has an average aspect ratio of, for example, 1.5 or greater, preferably 3 or greater, more preferably 5 or greater. The first powder may have an average aspect ratio of 7 or greater, 10 or greater, or 20 or greater. If the average aspect ratio is less than 1.5, adhesion to an application target (e.g., the skin) at the time of application may be impaired, which may lead to uneven application. The first powder may have an average aspect ratio of, for example, 200 or less, 170 or less, 150 or less, 120 or less, or 100 or less. Particularly, a platy powder may have an average aspect ratio of 20 or greater.

In cases where the powder-containing composition of the present disclosure is to be employed in an external-use skin preparation, examples of the first powder may include mica (including synthetic phlogopite), sericite (including calcined sericite), talc, or the like, whose surface has been coated with at least one selected from the group consisting of distearyldimonium chloride, magnesium stearate, triethoxysilylethyl polydimethylsiloxyethyl dimethicone, amodimethicone, hydrogen dimethicone, and zinc decyl trisiloxanecarboxylate.

The aspect ratio can be found, for example, by discretionarily extracting 100 particles and measuring each particle's length in the longer direction (planar direction) and length in the shorter direction (thickness direction) under microscopical observation, and calculating the ratio by dividing the longer-direction length by the shorter-direction length (i.e., “the longer-direction length/the shorter-direction length”). The longest length is to be chosen as the “longer-direction length”. The average aspect ratio may be an average value of the aspect ratios of the 100 particles extracted discretionarily.

For example, the first powder may be at least one selected from the group consisting of: talc, kaoline, sericite, muscovite, phlogopite, synthetic mica, synthetic fluorphlogopite, synthetic fluorphlogopite iron, lepidolite, biotite, calcined talc, calcined sericite, calcined muscovite, calcined phlogopite, vermiculite, magnesium carbonate, calcium carbonate, aluminum silicate, barium silicate, calcium silicate, magnesium silicate, strontium silicate, metal salts of tungstic acid, magnesium, silica, zeolite, barium sulfate, calcined calcium sulfate (calcined gypsum), calcium phosphate, fluorapatite, hydroxyapatite, ceramic powder, metallic soaps (e.g., zinc myristate, calcium palmitate, aluminum stearate, etc.), boron nitride, photochromic titanium oxide (titanium dioxide obtained by sintering of iron oxide), reduced zinc oxide; inorganic white pigments (e.g., titanium dioxide, zinc oxide, etc.); inorganic red pigments (e.g., iron oxide (colcothar), iron titanate, etc.); inorganic brown pigments (e.g., γ-iron oxide, etc.); inorganic yellow pigments (e.g., yellow iron oxide, loess, etc.); inorganic black pigments (e.g., black iron oxide, substoichiometric titanium oxide, etc.); inorganic purple pigments (e.g., mango violet, cobalt violet, etc.); inorganic green pigments (e.g., chromium oxide, chromium hydroxide, cobalt titanate, etc.); inorganic blue pigments (e.g., ultramarine blue, iron blue, etc.); pearl pigments (e.g., bismuth oxychloride, fish scale flake, titanated mica, iron oxide-coated titanated mica, substoichiometric titanium oxide-coated titanated mica, photochromic titanated mica, pigments employing substrates such as talc, glass, synthetic fluorphlogopite, silica, bismuth oxychloride, etc., instead of mica, pigments coated with substoichiometric titanium oxide, colored titanium oxide, iron oxide, alumina, silica, zirconia, zinc oxide, cobalt oxide, aluminum, etc., other than titanium oxide as the coating, functional pearl pigments in which, for example, the pearl pigment surface is coated with resin particles (Japanese Unexamined Patent Publication No. H11-92688), in which the pearl pigment surface is coated with aluminum hydroxide particles (Japanese Unexamined Patent Publication No. 2002-146238), in which the pearl pigment surface is coated with zinc oxide particles (Japanese Unexamined Patent Publication No. 2003-261421), and in which the pearl pigment surface is coated with barium sulfate particles (Japanese Unexamined Patent Publication No. 2003-61229), etc.); and metal powder pigments (e.g., aluminum powder, copper powder, etc.).

The average particle size of the first powder is preferably 0.01 μm or greater, more preferably 0.1 μm or greater, even more preferably 1 μm or greater. The average particle size of the first powder is preferably 200 μm or less, more preferably 100 μm or less, even more preferably 70 μm or less.

The average particle size of the first powder can be measured by employing dynamic light scattering.

It is preferred that the first powder is suppressed from aggregating, even in a state where the powder-containing composition is left to stand, and is dispersed within the powder-containing composition.

It is preferred that the content of the first powder relative to the mass of the powder-containing composition is preferably 0.5% by mass or greater, more preferably 1% by mass or greater, even more preferably 2% by mass or greater, even more preferably 3% by mass or greater. The content of the first powder relative to the mass of the powder-containing composition may be 4% by mass or greater, 5% by mass or greater, 6% by mass or greater, or 7% by mass or greater. If the content of the first powder is less than 0.5% by mass, a uniform luster cannot be achieved in the application region. It is preferred that the content of the first powder relative to the mass of the powder-containing composition is preferably 22% by mass or less, more preferably 20% by mass or less, even more preferably 18% by mass or less, even more preferably 16% by mass or less. The content of the first powder relative to the mass of the powder-containing composition may be 14% by mass or less, 12% by mass or less, 10% by mass or less, 8% by mass or less, 6% by mass or less, or 4% by mass or less. If the content of the first powder exceeds 22% by mass, powder dispersibility will deteriorate. Also, squeakiness is likely to arise at the time of application. Further, the uniformity of the coating film of the powder will deteriorate.

Liquid-State Polyol:

Examples of the liquid-state polyol may include glycerin, 1,3-butylene glycol, polyethylene glycol, diglycerin, dipropylene glycol, etc.

The content of the liquid-state polyol relative to the mass of the powder-containing composition is preferably 6% by mass or greater, more preferably 7% by mass or greater, even more preferably 8% by mass or greater. The content of the liquid-state polyol relative to the mass of the powder-containing composition may be 10% by mass or greater, 12% by mass or greater, or 15% by mass or greater. If the content of the liquid-state polyol is less than 6% by mass, it may be difficult to disperse the first powder within the composition. Also, squeakiness will arise at the time of application of the powder-containing composition. Furthermore, the luster in the application region where the powder-containing composition is applied will deteriorate. The content of the liquid-state polyol relative to the mass of the powder-containing composition is preferably 25% by mass or less, more preferably 22% by mass or less, even more preferably 20% by mass or less, even more preferably 16% by mass or less. The content of the liquid-state polyol relative to the mass of the powder-containing composition may be 14% by mass or less, or 12% by mass or less. If the content of the liquid-state polyol exceeds 25% by mass, stickiness will increase, and it will be difficult to apply the first powder uniformly.

In the present disclosure, “squeakiness” refers to a feel of lack of smoothness at the time of application, which is perceived when the composition is being applied to the skin. “Squeaky-powdery feel” refers to squeakiness ascribable to powder.

Water:

Any type of water used for cosmetics, quasi-pharmaceutical products, etc., may be used for the water. It is possible to use, for example, purified water, ion-exchanged water, tap water, etc.

The content of water relative to the mass of the powder-containing composition is preferably 50% by mass or greater, more preferably 55% by mass or greater, even more preferably 60% by mass or greater, more preferably 65% by mass or greater. The content of water relative to the mass of the powder-containing composition may be 70% by mass or greater, or 75% by mass or greater. If the content of water is less than 50% by mass, it will not be possible to obtain a favorable feel upon use. The content of water relative to the mass of the powder-containing composition is preferably 95% by mass or less, more preferably 90% by mass or less, even more preferably 85% by mass or less. The content of water relative to the mass of the powder-containing composition may be 80% by mass or less, 75% by mass or less, or 70% by mass or less.

Liquid-State Oily Component:

The liquid-state oily component is dispersed in an aqueous solvent (aqueous phase) including water. The liquid-state oily component can be dispersed in the aqueous phase without depending on a surfactant. It is thought that the liquid-state oily component is dispersed in the aqueous phase in a state of a giant emulsion. In a giant-emulsion state, the average particle size of the oil droplet particles may be from 5 μm to 30 μm. It is preferred that the liquid-state oily component is not separated from the aqueous phase.

Preferably, the liquid-state oily component is at least one selected from the group consisting of fluoro-oils, silicone oils, polar oils, and hydrocarbon oils. Examples of the liquid-state oily component may include dimethicone, hydrogenated polydecene, triethylhexanoin, ethylhexyl methoxycinnamate, etc.

Examples of oil-soluble UV absorbers may include: benzoic acid-based UV absorbers (e.g., para-aminobenzoic acid (abbreviated as PABA hereinbelow), PABA monoglycerin ester, N,N-dipropoxy PABA ethyl ester, N,N-diethoxy PABA ethyl ester, N,N-dimethyl PABA ethyl ester, N,N-dimethyl PABA butyl ester, N,N-dimethyl PABA ethyl ester, etc.); anthranilic acid-based UV absorbers (e.g., homomenthyl-N-acetylanthranilate, etc.); salicylic acid-based UV absorbers (e.g., amyl salicylate, menthyl salicylate, homomenthyl salicylate, octyl salicylate, phenyl salicylate, benzyl salicylate, p-isopropanol phenyl salicylate, homosalate, etc.); cinnamic acid-based UV absorbers (e.g., octyl methoxycinnamate, ethyl-4-isopropyl cinnamate, methyl-2,5-diisopropyl cinnamate, ethyl-2,4-diisopropyl cinnamate, methyl-2,4-diisopropyl cinnamate, propyl-p-methoxycinnamate, isopropyl-p-methoxycinnamate, isoamyl-p-methoxycinnamate, octyl-p-methoxycinnamate (2-ethylhexyl-p-methoxycinnamate, ethylhexyl methoxycinnamate), 2-ethoxy ethyl-p-methoxy cinnamate, cyclohexyl-p-methoxycinnamate, ethyl-α-cyano-β-phenyl cinnamate, 2-ethylhexyl-α-cyano-β-phenyl cinnamate, glyceryl mono-2-ethylhexanoyl-di-para-methoxy cinnamate, etc.); 3-(4 ‘-methylbenzylidene)-d,l-camphor, 3-benzylidene-d,l-camphor; 2-phenyl-5-methylbenzoxazole; 2,2’-hydroxy-5-methylphenyl benzotriazole; 2-(2′-hydroxy-5′-t-octylphenyl)benzotriazole; 2-(2′-hydroxy-5′-methylphenyl)benzotriazole; dibenzalazine; dianisoylmethane; 4-methoxy-4′-t-butyldibenzoylmethane; 5-(3,3-dimethyl-2-norbornylidene)-3-pentan-2-one, dimorpholinopyridazinone; 2-ethylhexyl-2-cyano-3,3-diphenyl acrylate (octocrylene); 2,4-bis-{[4-(2-ethylhexyloxy)-2-hydroxy]-phenyl}-6-(4-methoxyphenyl)-(1,3,5)-triazine; and benzophenone-based UV absorbers (e.g., 2,4-dihydroxybenzophenone, 2,2′-dihydroxy-4-methoxybenzophenone, 2,2′-dihydroxy-4,4′-dimethoxybenzophenone, 2,2′,4,4′-tetrahydroxybenzophenone, 2-hydroxy-4-methoxybenzophenone, 2-hydroxy-4-methoxy-4′-methylbenzophenone, 2-hydroxy-4-methoxybenzophenone-5-sulfonic acid salt, 4-phenylbenzophenone, 2-ethylhexyl-4′-phenyl-benzophenone-2-carboxylate, 2-hydroxy-4-n-octoxybenzophenone, 4-hydroxy-3-carboxybenzophenone, etc.).

The content of the liquid-state oily component relative to the mass of the powder-containing composition is preferably 3% by mass or greater, more preferably 5% by mass or greater. The content of the liquid-state oily component relative to the mass of the powder-containing composition may be 6% by mass or greater, or 7% by mass or greater. If the content of the liquid-state oily component is less than 3% by mass, the user may not be able to feel moistness. The content of the liquid-state oily component relative to the mass of the powder-containing composition is preferably 12% by mass or less, more preferably 10% by mass or less. The content of the liquid-state oily component relative to the mass of the powder-containing composition may be 8% by mass or less, or 6% by mass or less. If the content of the liquid-state oily component exceeds 12% by mass, stickiness will arise.

Other Oily Components:

The powder-containing composition of the present disclosure may contain oily components other than the aforementioned liquid-state oily component. Examples of oily components that may be used include waxes, liquid oils, solid fats, wax esters, hydrocarbons, higher fatty acids, higher alcohols, synthetic ester oils, silicone oils, etc.

Examples of the waxes that may be used may include beeswax, candelilla wax, cotton wax, carnauba wax, bayberry wax, insect wax, spermaceti, montan wax, bran wax, lanolin, kapok wax, lanolin acetate, liquid lanolin, sugarcane wax, lanolin fatty acid isopropyl ester, hexyl laurate, reduced lanolin, jojoba wax, hardened lanolin, shellac wax, POE lanolin alcohol ether, POE lanolin alcohol acetate, POE cholesterol ether, lanolin fatty acid polyethylene glycol, POE hydrogenated lanolin alcohol ether, liquid paraffin, ozocerite, squalane, pristane, paraffin, ceresin. squalene, vaseline, microcrystalline wax, Fischer-Tropsch was, for example, and the like.

Examples of the liquid oil that may be used may include avocado oil, camellia oil, turtle oil, macadamia nut oil, corn oil, mink oil, olive oil, rapeseed oil, egg yolk oil, sesame oil, par chic oil, wheat germ oil, southern piece oil, castor oil, linseed oil, safflower oil, cotton seed oil, perilla oil, soybean oil, groundnut oil, brown real oil, torreya oil, rice bran oil, Chinese tung oil, Japanese tung oil, jojoba oil, germ oil, triglycerol, and the like.

Examples of the solid fat that may be used may include cacao butter, coconut oil, horse fat, hydrogenated coconut oil, palm oil, beef tallow, sheep tallow, hydrogenated beef tallow, palm kernel oil, lard, beef bones fat, Japan wax kernel oil, hardened oil, hoof oil, Japan wax, hydrogenated caster oil, and the like.

Examples of the waxes that may be used may include beeswax, candelilla wax, cotton wax, carnauba wax, bayberry wax, insect wax, spermaceti, montan wax, bran wax, lanolin, kapok wax, lanolin acetate, liquid lanolin, sugarcane wax, lanolin fatty acid isopropyl ester, hexyl laurate, reduced lanolin, jojoba wax, hardened lanolin, shellac wax, POE lanolin alcohol ether, POE lanolin alcohol acetate, POE cholesterol ether, lanolin fatty acid polyethylene glycol, POE hydrogenated lanolin alcohol ether, and the like.

Examples of the hydrocarbon oils that may be used may include liquid paraffin, ozocerite, squalane, pristane, paraffin, ceresin. squalene, vaseline, microcrystalline wax, and the like.

Examples of the higher fatty acid that may be used may include lauric acid, myristic acid, palmitic acid, stearic acid, behenic acid, oleic acid, undecylenic acid, tallic acid, isostearic acid, linoleic acid, linolenic acid, eicosapentaenoic acid(EPA), docosahexaenoic acid(DHA) and the like.

Examples of the higher alcohol that may be used may include linear alcohol (such as lauryl alcohol, cetyl alcohol, stearyl alcohol, behenyl alcohol, myristyl alcohol, oleyl alcohol, and cetostearyl alcohol); branched-chain alcohol (such as monostearylglycerin ether (batyl alcohol), 2-decyltetradecinol, lanolin alcohol, cholesterol, phytosterol, hexyldodecanol, isostearyl alcohol, and octyldodecanol) and the like.

Examples of the synthesis ester oils that may be used may include isopropyl myristate, cetyl octanoate, octyldodecyl myristate, isopropyl palmitate, butyl stearate, hexyl laurate, myristyl myristate, decyl oleate, hexyldecyl dimethyl octanoate, cetyl lactate, myristyl lactate, lanolin acetate, isocetyl stearate, isocetyl isostearate, cholesteryl 12-hydroxy stearate, ethylene glycol di-2-ethyl hexanoate, di-penta erythritol fatty acid ester, N-alkyl glycol monoisostearate, neopentyl glycol dicaprate, diisostearyl malate, glyceryl di-2-heptyl undecanoate, trimethyrol propane tri-2-ethyl hexanoate, trimethyrol propane triisostearate, pentaerythritol tetra-2-ethyl hexanoate, glyceryl tri-2-ethyl hexanoate, glyceryl trioctanoate, glyceryl triisopalmitate, trimethyrol propane triisostearate, cetyl 2-ethylhexanoate, 2-ethylhexyl palmitate, glyceryl trimyristate, glyceride tri-2-heptyl undecanoate, castor oil fatty acid methyl ester, oleyl oleate, acetoglyceride, 2-heptylundecyl palmitate, diisobutyl adipate, N-lauroyl-L-glutamic acid-2-octyldodecyl ester, di-2-heptylundecyl adipate, ethyl laurate, di-2-ethylhexyl sebacate, 2-hexyldecyl myristate, 2-hexyldecyl palmitate, 2-hexyldecyl adipate, diisopropyl sebacate, 2-ethylhexyl succinate, triethyl citrate, and the like.

Examples of the silicone oil may include silicone compounds such as dimethylpolysiloxane, methylhydrogenpolysiloxane, methylphenylpolysiloxane, stearoxymethylpolysiloxane, polyether-modified organopolysiloxane, fluoroalkyl/polyoxyalkylene co-modified organopolysiloxane, alkyl-modified organopolysiloxane, terminal-modified organopolysiloxane, fluorine-modified organopolysiloxane, amino-modified organopolysiloxane, silicone gel, acrylic silicone, trimethylsiloxysilicic acid, silicone RTV rubber, cyclopentasiloxane and the like.

Thickener:

The powder-containing composition of the present disclosure may further contain a thickener. The type of thickener is not particularly limited. The thickener can be added to achieve a desired viscosity depending on the purpose.

Examples of the thickener may include hydrophobic polymers. Examples of hydrophobic polymers may include hydrophobically modified polyether urethanes. Examples of hydrophobically modified polyether urethanes may be selected from at least one selected from the group consisting of PEG-240/HDI copolymer bis-decyltetradeceth-20 ether and polyurethane-59. Particularly, PEG-240/HDI copolymer bis-decyltetradeceth-20 ether is preferable. PEG-240/HDI copolymer bis-decyltetradeceth-20 ether is a hydrophobically modified polyether urethane consisting of a copolymer of PEG-240, decyltetradeceth-20, and hexamethylene diisocyanate (HDI). An example of a commercially available product of PEG-240/HDI copolymer bis-decyltetradeceth-20 ether includes Adeka Nol (registered trademark) GT-700 (from ADEKA Corporation). Polyurethane-59 is a hydrophobically modified polyether urethane consisting of a copolymer of ethylhexylglycerin, PEG-240, tetradecyloctadeceth-100, and hexamethylene diisocyanate (HDI). An example of a commercially available product of polyurethane-59 includes Adeka Nol (registered trademark) GT-930 (from ADEKA Corporation).

Examples of other thickeners that may be used include acrylamide-based thickeners such as dimethylacrylamide/sodium acryloyldimethyltaurate crosspolymer, etc.

Examples of the thickeners other than the aforementioned thickeners may include gum arabic, carrageenan, karaya gum, tragacanth gum, carob gum, quince seed (marmelo), casein, dextrin, gelatin, sodium pectate, sodium alginate, methyl cellulose, ethyl cellulose, carboxymethyl cellulose (CMC), hydroxyethyl cellulose, hydroxypropyl cellulose, polyvinyl alcohol (PVA), polyvinylmethyl ether (PVM), PVP (polyvinyl pyrrolidone), polysodium acrylate, carboxyvinyl polymer, locust bean gum, guar gum, tamarind gum, dialkyldimethylammonium sulfate cellulose, xanthan gum, aluminum magnesium silicate, bentonite, hectorite, aluminum magnesium silicate (Veegum), sodium magnesium silicate (Laponite), silicic acid anhydride, taurate-based synthetic polymers, and acrylate-based synthetic polymers.

The content of the thickener relative to the mass of the powder-containing composition may be 0.2% by mass or greater, or 0.5% by mass or greater. The content of the thickener relative to the mass of the powder-containing composition may be 2% by mass or less, or 1.5% by mass or less.

Second Powder:

The powder-containing composition of the present disclosure may contain a second powder, which is a spherical powder, and the content thereof relative to 1 part by mass of the first powder is preferably 1.5 parts by mass or less, more preferably 1 part by mass or less, even more preferably 0.5 parts by mass or less, even more preferably 0.1 parts by mass or less. It is further preferred that the second powder is not contained in the powder-containing composition (i.e., the content is 0% by mass). If the content of the second powder exceeds 1.5 parts by mass, adhesion of the first powder to an application target (e.g., the skin) may be impaired, which may lead to uneven application and deterioration in uniform luster.

In the present disclosure, “spherical powder” refers to powder constituted mainly by spherical particles. Powder having an average aspect ratio of less than 1.2 may be referred to as “spherical powder”. The spherical powder can be verified, for example, by microscopical observation.

Examples of the second powder may include inorganic spherical powders (e.g., silica, alumina, titania, calcium carbonate) and organic spherical powders (e.g., polyamide resin powder (nylon powder), polyethylene powder, polymethyl methacrylate powder, polystyrene powder, styrene/acrylic acid copolymer resin powder, benzoguanamine resin powder, polytetrafluoroethylene powder, cellulose powder, silicone resin powder, silk powder, wool powder, urethane powder, etc.).

The average particle size of the second powder is preferably 0.1 μm or greater, more preferably 1 μm or greater, even more preferably 5 μm or greater. The average particle size of the second powder is preferably 50 μm or less, more preferably 20 μm or less, even more preferably 15 μm or less.

The average particle size of the second powder can be measured by employing dynamic light scattering.

Surfactant:

The powder-containing composition of the present disclosure may contain a surfactant, and the content thereof relative to the mass of the powder-containing composition is 0.5% by mass or less, preferably 0.3% by mass or less, more preferably 0.1% by mass or less, and even more preferably, no substantial amount is contained (i.e., the content is 0% by mass). By containing 0.5% by mass or less of the surfactant, stickiness upon application to the skin can be suppressed, and also, stimulation to the skin can be reduced.

Examples of the anionic surfactants that may be used may include fatty acid soap (such as sodium laurate, and sodium palmitate); higher alkyl sulfate ester salt (such as sodium lauryl sulfate, and potassium lauryl sulfate); alkyl ether sulfate ester salt (such as POE-lauryl sulfate triethanolamine, and sodium POE-lauryl sulfate); N-acyl sarcosinic acid (such as sodium lauroyl sarcocinate); higher fatty acid amide sulfonate (such as sodium N-stearoyl-N-methyltaurate, sodium N-myristoyl-N-methyltaurate, sodium methyl cocoyl taurate, and sodium laurylmethyl taurate); phosphate ester salt (sodium POE-oleylether phosphate, POE-stearylether phosphate, potassium cetyl phosphate); sulfosuccinate (such as sodium di-2-ethylhexyl sulfosuccinate, sodium monolauroyl monoethanolamide polyethylene sulfosuccinate, and sodium lauryl polypropylene glycol sulfosuccinate); alkylbenzene sulfonate (such as sodium linear dodecylbenzene sulfonate, triethanolamine linear dodeylbenzene sulfonate, and linear dodecylbenzene sulfonate); higher fatty acid ester sulfate ester salt (such as sodium hydrogenated gryceryl cocoate sulfate); N-acyl glutamate (such as monosodium N-lauroyl glutamate, disodium N-stearoyl glutamate, and monosodium N-myristoyl-L-glutamate); sulfonated oil (such as Turkey red oil); POE-alkyl ether carboxylic acid; POE-alkyl aryl ether carboxylate; α-olefine sulfonate; higher fatty acid ester sulfonate; secondary alcohol sulfate ester salt; higher fatty acid alkylolamide sulfate ester salt; sodium lauroyl monoethanolamide succinate; N-palmitoyl asparaginate ditriethanolamine; sodium casein; and the like.

Examples of the cationic surfactants may include alkyltrimethyl ammonium salt (such as stearyltrimethyl ammonium chloride, lauryltrimethyl ammonium chloride); alkylpyridinium salt (such as cetylpyridinium chloride); dialkyldimethyl ammonium salt (such as distearyldimethyl ammonium chloride); poly (N,N′-dimethyl-3,5-methylenepiperidinium) chloride; alkyl quaternary ammonium salt; alkyldimethylbenzyl ammonium salt; alkylisoquinolinium salt; dialkylmorphonium salt; POE alkylamine; alkylamine salt; polyamine fatty acid derivative; amyl alcohol fatty acid derivative; benzalkonium chloride; benzethonium chloride, and the like.

Examples of the amphoteric surfactant that may be used may include: imidazoline-based amphoteric surfactant (such as sodium 2-undecyl-N,N,N-(hydroxyethylcarboxymethyl)-2-imidazoline and 2-cocoyl-2-imidazolinium hydroxide-1-carboxyethyloxy disodium salt); and betaine-based surfactant (such as 2-heptadecyl-N-carboxymethyl-N-hydroxyethyl imidazolinium betaine, lauryl dimethylaminoacetic acid betaine, alkyl betaine, amidobetaine, and sulfobetaine).

Examples of the hydrophilic nonionic surfactants that may be used may include POE sorbitan fatty acid ester (such as POE sorbitan monooleate, POE sorbitan monostearate, POE sorbitan monooleate, POE sorbitan tetraoleate); POE sorbit fatty acid ester (such as POE sorbit monolaurate, POE sorbit monooleate, POE sorbit pentaoleate, POE sorbit monostearate), POE glyceryl fatty acid ester (such as POE monooleate such as POE glyceryl monostearate, POE glyceryl monoisostearate, POE glyceryl triisostearate); POE fatty acid ester (such as POE distearate, POE monodioleate, ethyleneglycol distearate); POE alkyl ether (such as POE lauryl ether, POE oleyl ether, POE stearyl ether, POE behenyl ether, POE-2-octyldodecyl ether, POE cholestanol ether); puluronic type (such as Puluronic), POE/POP alkyl ethers (such as POE/POP cetyl ether, POE/POP 2-decyltetradecyl ether, POE/POP monobutyl ether, POE/POP hydrogenated lanoline, POE/POP glycerin ether); tetra POE/tetra POP ethylenediamine condensation products (such as Tetronic); POE castor oil hydrogenated castor oil derivative (such as POE caster oil, POE hydrogenated caster oil, POE hydrogenated caster oil monoisostearate, POE hydrogenated castor oil triisostearate, POE hydrogenated caster oil monopyroglutamate monoisostearate diester, POE hydrogenated oil maleate); POE beeswax/lanoline derivative (such as POE sorbitol beeswax); alkanolamide (such as coconut oil fatty acid diethanolamide, lauric acid monoethanolamide, fatty acid isopropanolamide); POE propyleneglycol fatty acid ester; POE alkyl amines; POE fatty acid amide; sucrose fatty acid ester; alkylethoxydimethylamine oxide; trioleyl phosphoric acid and the like.

Examples of the lipophilic nonionic surfactants may include sorbitan fatty acid ester (such as sorbitan monooleate, sorbitan monoisostearate, sorbitan monolaurate, sorbitan monopalmitate, sorbitan monostearate, sorbitan sesquioleate, sorbitan trioleate, diglycerol sorbitan penta-2 ethylhexylate, diglycerol sorbitan tetra-2 ethylhexylate, etc); glyceryl polyglyceryl fatty acid (such as glyceryl monocotton oil fatty acid, glyceryl monoerucate, glyceryl sesquioleate, glyceryl monostearate, glyceryl α, α′-oleate pyroglutamate, glyceryl monostearate malate, etc); propylene glycol fatty acid ester (such as propylene glycol monostearate, etc); hydrogenated caster oil derivative; glyceryl alkyl ether, and the like.

Viscosity:

It is preferred that the powder-containing composition of the present disclosure has a viscosity of 1,000 mPa s or greater, preferably 5,000 mPa s or greater, more preferably 10,000 mPa·s or greater. If the viscosity is less than 1,000 mPa·s, powder dispersibility cannot be maintained over time. The viscosity of the powder-containing composition of the present disclosure is preferably 150,000 mPa·s or less, more preferably 100,000 mPa s or less. If the viscosity exceeds 150,000 mPa s, easy applicability to the skin will deteriorate. The viscosity can be measured with a Brookfield viscometer (spindle No. 1; rotation speed: 60 rpm) at 30° C.

Others:

If necessary, the powder-containing composition of the present disclosure may contain other components as appropriate, such as water-soluble alcohols, moisturizers, film-forming agents, oil-soluble UV absorbers, water-soluble UV absorbers, metal ion sequestering agents, amino acids, organic amines, polymer emulsions, pH adjusters, skin nutrients, vitamins, antioxidants, antioxidant aids, perfumes, etc., in amounts that do not inhibit the effects of the present disclosure.

Examples of water-soluble alcohols may include at least one type selected from lower alcohols, polyhydric alcohols, polyhydric alcohol polymers, dihydric alcohol alkyl ethers, dihydric alcohol alkyl ethers, dihydric alcohol ether esters, glycerin monoalkyl ethers, sugar alcohols, monosaccharides, oligosaccharides, polysaccharides, and derivatives of the above.

Examples of the lower alcohol may include ethanol, propanol, isopropanol, isobutyl alcohol, t-butyl alcohol, and the like.

Examples of the polyol may include dihydric alcohol (such as ethylene glycol, propylen glycol, trimethylene glycol, 1,2-butylene glycol, 1,3-butylene glycol, tetramethylene glycol, 2,3-butylene glycol, pentamethylene glycol, 2-butene-1,4-diol, hexylene glycol, octylene glycol, etc); trihydric alcohol (such as glycerin, trimethylolpropane, etc); tetrahydric alcohol (such as such as pentaerythritol such as 1,2,6-hexanetriol, etc); pentahydric alcohol (such as xylitol, etc); hexahydric alcohol (such as sorbitol, mannitol, etc); polyhydric alcohol polymer (such as diethylene glycol, dipropylene glycol, triethylene glycol, polypropylene glycol, tetraethylene glycol, diglycerin, polyethylene glycol, triglycerin, tetraglycerin, polyglycerin, etc); dihydric alcohol alkyl ethers (such as ethylene glycol monomethyl ether, ethylene glycol monoethyl ether, ethylene glycol monobutyl ether, ethylene glycol monomphenyl ether, ethylene glycol monohexyl ether, ethylene glycol mono2-methylhexyl ether, ethylene glycol isoamyl ether, ethylene glycol benzil ether, ethylene glycol isopropyl ether, ethylene glycol dimethyl ether, ethylene glycol diethyl ether, ethylene glycol dibutyl ether, etc); dihydric alcohol alkyl ethers (such as diethylene glycol monomethyl ether, diethylene glycol monoethyl ether, diethylene glycol monombutyl ether, diethylene glycol dimethyl ether, diethylene glycol diethyl ether, diethylene glycol butyl ether, diethylene glycol methylethyl ether, triethylene glycol monomethyl ether, triethylene glycol monoethyl ether, propylene glycol monomethyl ether, propylene glycol monoethyl ether, propylene glycol monobutyl ether, propylene glycol isopropyl ether, dipropylene glycol methyl ether, dipropylene glycol ethyl ether, dipropylene glycol butyl ether, etc); dihydric alcohol ether ethers (such as ethylene glycol monomethyl ether acetate, ethylene glycol monoethyl ether acetate, ethylene glycol monobutyl ether acetate, ethylene glycol monophenyl ether acetate, ethylene glycol diadipate, ethylene glycol disaccinate, diethylene glycol monoethyl ether acetate, diethylene glycol monobutyl ether acetate, propylene glycol monomethyl ether acetate, propylene glycol monoethyl ether acetate, propylene glycol monopropyl ether acetate, propylene glycol monophenyl ether acetate, etc); glycerin monoalkyl ether (such as chimyl alcohol, selachyl alcohol, batyl alcohol, etc); sugar alcohol (such as sorbitol, maltitol, maltotriose, mannitol, sucrose, erythritol, glucose, fructose, starch sugar, maltose, xylitol, starch sugar hydrogenated alcohol, etc); glycolide, tetrahydrofurfuryl alcohol; POE-tetrahydrofurfuryl alcohol; POP/POE-butyl ether; tripolyoxypropylene glycerin ether; POP-glycerin ether; POP-glycerin ether phosphoric acid; POP/POE-pentaerythritol ether; polyglycerin, and the like.

Examples of the monosaccharides may include at least one selected from triose (such as D-glyceryl aldehyde, dihydroxyacetone, etc); tetrose (such as D-erythrose, D-erythrulose, D-threose, erythritol, etc); pentaose (such as L-arabinose, D-xylose, L-lyxose, D-arabinose, D-ribose, D-ribulose, D-xylulose, L-xylulose, etc); hexalose (such as D-glucose, D-talose, D-psicose, D-galactose, D-fructose, L-galactose, L-mannose, D-tagatose, etc); heptose (such as aldoheptose, heptulose, etc); octose (such as octulose, etc); deoxy sugar (such as 2-deoxy-D-ribose, 6-deoxy-L-galactose, 6-deoxy-L-mannose, etc); amino sugar (such as D-glucosamine, D-galactosamine, sialic acid, amino uronic acid, muramic acid, etc); uronic acid (such as D-grucuronic acid, D-mannuronic acid, L-guluronic acid, D-garacturonic acid, L-iduronic acid, etc) and the like.

Examples of the oligosaccharide may include at least one selected from sucrose, guntianose, umbelliferose, lactose, planteose, is olignose s, α,α-trehalose, raffinose, lignoses, umbilicin, stachyose, verbascoses, and the like.

Examples of the polysaccharide may include at least one selected from cellulose, quince seed, chondroitinsulfate, starch, galactan, dermatan sulfate, glycogen, acasia gum, heparansulfate, hyaluronan, gum tragacanth, keratan sulfate, chondoroitin, xanthan gum, mucoitin sulfate, guar gum, dextran, keratosulfate, locust bean gum, succinoglycan, caronic acid, and the like.

Examples of other polyols may include at least one polyol selected from polyoxyethylene methyl glucoside (Glucam E-10) and polyoxypropylene methyl glucoside (Glucam P-10).[0085]

Examples of the natural water-soluble polymer may include plant-based polymer (such as gum Arabic, gum tragacanth, galactan, guar gum, locust bean gum, gum karaya, carrageenan, pectine, agar, quince seed (cydonia oblonga), algae colloid (brown algae extract), starch (rice, corn, potato, wheat), glicyrrhizic acid); microorganism based polymer (such as xanthan gum, dextran, succinoglycan, pullulan, etc), animal-based polymer (such as collagen, casein, albumin, gelatine, etc) and the like.

Examples of the semisynthetic water-soluble polymer may include starch-based polymer (such as carboxymethyl starch, methylhydroxypropyl starch, etc); cellulose-based polymer (such as methylcellulose, ethylcellulose, methylhydroxypropylcellulose, hydroxyethylcellulose, cellulose sodium sulfate, hydroxypropylcellulose, carboxymethylcellulose, sodium calboxymethyl cellulose, crystalline cellulose, cellulose powder, etc); algin acid-based polymer (such as sodium alginate, propylene glycol alginate ester, etc), and the like.

Examples of the moisturizers may include polyethylene glycol, propylene glycol, glycerin, 1,3-butylene glycol, xylitol, sorbitol, maltitol, chondroitin sulfate, hyaluronic acid, mucoitin sulfate, charonic acid, atelocollagen, cholesteryl 12-hydroxystearate, sodium lactate, bile salt, dl-pyrrolidone carboxylate, alkyleneoxide derivative, short-chain soluble collagen, diglycerin (EO)PO adduct, chestnut rose extract, yarrow extract, melilot extract, and the like.

Examples of the film-forming agent may include an anionic film-forming agent (such as (meta)acrylic acid/(meta)acrylic acid ester copolymer, methyl vinyl ether/maleic anhydride coplymer, etc), a cationic film-forming agent (such as cationic cellulose, diallyldimethylammonium chloride polymer, diallyldimethylammonium chloride/acrylic amide copolymer, etc), a nonionc film-forming agent (such as polyvinyl alcohol, polyvinylpyrrolidone, polyvinyl acetate, polyacrylic ester copolymer, (meta)acrylamide, polymeric silicone, silicone resin, trimethylsiloxysilicate, etc), and the like. Polyvinyl acetate is particularly preferable as the film-forming agent.

Examples of water-soluble UV absorbers may include: benzophenone-based UV absorbers (e.g. 2-hydroxy-4-methoxybenzophenone-5-sulfate, etc.); benzylidene camphor-based UV absorbers (e.g. benzylidene camphor sulfonic acid, terephthalylidene dicamphor sulfonic acid, etc.); phenylbenzimidazole-based UV absorbers (e.g. phenylbenzimidazole sulfonic acid, etc.)

Examples of the metal ion sequestrant may include 1-hydroxyethane-1, 1-diphosphonic acid, 1-hydroxyethane, 1-diphosphonic acid 4Na salt, disodium edetate, trisodium edetate, tetrasodium edetate, sodium citrate, sodium polyphosphate, sodium metaphosphate, gluconic acid, phosphoric acid, citric acid, ascorbic acid, succinic acid, edetic acid, trisodium hydroxyethyl ethylenediamine triacetate, and the like.

Examples of the amino acid may include neutral amino acid (such as threonine, cysteine, etc); basic amino acid (such as hydroxylysine, etc) and the like. Examples of the amino acid derivative may include sodium acyl sarcosinate (sodium lauroyl sarcosinate), acyl glutamate, sodium acyl 0-alanine, glutathione, pyrrolidone carboxylate, and the like.

Examples of the organic amine may include monoethanolamine, diethanolamine, triethanolamine, morpholine, triisopropanolamine, 2-amino-2-methyl-1,3-propanediol, 2-amino-2-methyl-1-propanol, and the like.

Examples of the polymer emulsion may include acrylic resin emulsion, ethyl polyacrylate emulsion, solution of acrylic resin, polyacrylalkylester emulsion, polyvinyl acetate resin emulsion, natural rubber latex, and the like.

Examples of the pH modifier may include buffer such as lactic acid-sodium lactate, citric acid-sodium citrate, succinic acid-sodium succinate, and the like.

Examples of the vitamins may include vitamine A, B1, B2, B6, C, E and derivatives thereof, pantothenic acid and derivatives thereof, biotin, and the like.

Examples of the anti-oxidant may include tocopherols, dibutyl hydroxy toluene, butyl hydroxy anisole, and gallic acid esters, and the like.

Examples of the anti-oxidant aid may include phosphoric acid, citric acid, ascorbic acid, maleic acid, malonic acid, succinic acid, fumaric acid, cephalin, hexamethaphosphate, phytic acid, ethylenediaminetetraacetic acid, and the like.

Examples of other containable compositions may include an antiseptic agent (such as ethylparaben, butylparaben, chlorphenesin, 2-phenoxyethanol, etc); antiphlogistic (such as glycyrrhizinic acid derivatives, glycyrrhetic acid derivatives, salicylic acid derivatives, hinokitiol, zinc oxide, allantoin, etc); a skin-whitening agent (such as placental extract, saxifrage extract, arbutin, etc); various extracts (such as phellodendron bark (cork tree bark), coptis rhizome, lithospermum, peony, swertia herb, birch, sage, loquat, carrot, aloe, mallow, iris, grape, coix seed, sponge gourd, lily, saffron, cnidium rhizome, ginger, hypericum, restharrow, garlic, red pepper, citrus unshiu, Japanese angelica, seaweed, etc); an activator (such as royal jelly, photosenstizer, cholesterol derivatives, etc); a blood circulation promotion agent (such as nonylic acid vanillylamide, nicotine acid benzyl ester, nicotine acid β-butoxyethyl ester, capsaicin, zingerone, cantharides tincture, ichthammol, tannic acid, α-borneol, tocopheryl nicotinate, meso-inositol hexanicotinate, cyclandelate, cinnarizine, tolazoline, acetylcholine, verapamil, cepharanthine, γ-oryzanol, etc); an antiseborrheric agent, (such as sulfur, thianthl, etc); an anti-inflammatory agent (such as tranexamic acid, thiotaurine, hypotaurine, etc), and the like.

The composition of the present disclosure further may include, as necessary, caffeine, tannin, verapamil, tranexamic acid and derivatives thereof; various crude drug extracts such as licorice, Chinese quince, Pyrola japonica and the like; drugs such as tocopherol acetate, glycyrrhetinic acid, glycyrrhizic acid and derivatives thereof, or salts thereof; skin-whitening agents such as vitamin C, magnesium ascorbyl phosphate, ascorbic acid glucoside, arbutin, kojic acid and the like; amino acids such as arginine and lysine and the like and derivatives thereof.

The powder-containing composition of the present disclosure is applicable, for example, to eye shadows, base cosmetics (base makeup), veil cosmetics (foundations), mascaras, etc.

Manufacturing Method:

A method for manufacturing the powder-containing composition of the present disclosure will be described. The powder-containing composition of the present disclosure can be manufactured according to a known method. For example, a first powder and a liquid-state polyol are added to water and are mixed. Then, to this mixture, an oil component is added in small amounts while mixing at low speed. In cases where the powder-containing composition is to contain a thickener, it is preferred to first mix the water and the thickener. In this way, the powder-containing composition of the present disclosure can be manufactured.

There may be cases where it is difficult, or utterly impractical, to directly define the powder-containing composition of the present disclosure based on the compositional makeup, structure, etc., thereof. In such circumstances, it should be permissible to define the powder-containing composition of the present disclosure according to methods for producing the same.

In the powder-containing composition of the present disclosure, aggregation of the first powder is suppressed, and the first powder is dispersed within the composition. Thus, the user can apply the first powder uniformly.

The powder-containing composition of the present disclosure allows the first powder to be applied uniformly to an application target (e.g., the skin). Stated differently, the powder-containing composition of the present disclosure can suppress uneven application. Thus, the appearance of the application region can be improved. Further, by enabling the powder to adhere uniformly to an application target (e.g., the skin), secondary adhesion to masks, clothing, etc., can be suppressed.

With the powder-containing composition of the present disclosure, squeakiness caused by powder arising at the time of application can be suppressed. Thus, the user can perceive a pleasant feeling upon application.

With the powder-containing composition of the present disclosure, due to the liquid-state oily component, the user can feel moistness when the powder-containing composition is applied to the skin.

The powder-containing composition of the present disclosure has a low surfactant content. Thus, with the powder-containing composition of the present disclosure, stickiness at the time of application can be suppressed. In cases where the composition is applied to the skin, stimulation to the skin can be reduced.

The powder-containing composition of the present disclosure can provide luster to the application region where the composition is applied.

### EXAMPLES

The powder-containing composition of the present disclosure will be described below by way of examples. The powder-containing composition of the present disclosure is, however, not limited to the following examples. The content by percentage of each of the components shown in the Tables is in terms of percent by mass (mass %).

### Test Examples 1 to 31

Powder-containing compositions according to Test Examples 1 to 31 shown in Table 1 were prepared. Each powder-containing composition was tested in terms of dispersibility, moistness, lack of squeakiness, uniformity of the coating film, lack of stickiness, and shine on the skin. The evaluation criteria for these test items are described below. Tables 1 to 7 show the compositional makeup and evaluation for each of the powder-containing compositions.

Dispersibility:

The powder-containing compositions of the respective test examples were left to stand for 10 days, to examine the dispersibility of powder.
A: Powder was stably dispersed without aggregating, even after 10 days.
B: A miniscule amount of powder aggregated after 10 days.
C: A small amount of powder aggregated after 10 days.
D: Powder aggregated in a day.
E: Powder aggregated immediately after being left to stand.

Lack of Squeakiness:

Ten expert panelists applied each of the powder-containing compositions of the respective test examples to their arm, and evaluated whether they felt squeakiness at the time of application. Each evaluation item was rated as follows according to the number of panelists.
A: Two or fewer panelists felt squeakiness at the time of application.
B: Three to four panelists felt squeakiness at the time of application.
C: Five to six panelists felt squeakiness at the time of application.
D: Seven to eight panelists felt squeakiness at the time of application.
E: Nine or more panelists felt squeakiness at the time of application.

Uniformity of Application:

Ten expert panelists applied each of the powder-containing compositions of the respective test examples to their arm, and evaluated whether it was possible to uniformly apply the powder onto the skin. Each evaluation item was rated as follows according to the number of panelists.
A: Nine or more panelists felt that uniform application was possible.
B: Seven to eight panelists felt that uniform application was possible.
C: Five to six panelists felt that uniform application was possible.
D: Three to four panelists felt that uniform application was possible.
E: Two or fewer panelists felt that uniform application was possible.

Lack of Stickiness:

Ten expert panelists applied each of the powder-containing compositions of the respective test examples to their arm, and evaluated whether there was no stickiness in the application region. Each evaluation item was rated as follows according to the number of panelists.
A: Nine or more panelists felt no stickiness.
B: Seven to eight panelists felt no stickiness.
C: Five to six panelists felt no stickiness.
D: Three to four panelists felt no stickiness.
E: Two or fewer panelists felt no stickiness.

Shine in Application Region:

Ten expert panelists applied each of the powder-containing compositions of the respective test examples to their arm, and evaluated whether there was luster in the application region. Each evaluation item was rated as follows according to the number of panelists.
A: Nine or more panelists felt that there was luster.
B: Seven to eight panelists felt that there was luster.
C: Five to six panelists felt that there was luster.
D: Three to four panelists felt that there was luster.
E: Two or fewer panelists felt that there was luster.

Moistness:

Ten expert panelists applied each of the powder-containing compositions of the respective test examples to their arm, and evaluated whether there was moistness after application. Each evaluation item was rated as follows according to the number of panelists.
A: Nine or more panelists felt that there was moistness.
B: Seven to eight panelists felt that there was moistness.
C: Five to six panelists felt that there was moistness.
D: Three to four panelists felt that there was moistness.
E: Two or fewer panelists felt that there was moistness.

### Test Examples 1 to 6

Powder-containing compositions were prepared by changing the content by percentage of the liquid-state polyol. Powder dispersibility deteriorated when the content of the liquid-state polyol was 5% by mass, and also, the number of panelists that felt squeakiness increased, and the number of panelists that perceived luster in the application region decreased. From the above, it is thought that the content of the liquid-state polyol is preferably 6% by mass or greater, more preferably 8% by mass or greater, even more preferably 10% by mass or greater.

A good rating was obtained even when the content of the liquid-state polyol was 25% by mass. It was possible to further improve the rating when the content of the liquid-state polyol was 20% by mass or less, preferably 16% by mass or less.

TABLE 1

| Test Example | | 1 | 2 | 3 | 4 | 5 | 6 |
|---|---|---|---|---|---|---|---|
| Distearyldimonium chloride- | | 2.879 | 2.879 | 2.879 | 2.879 | 2.879 | 2.879 |
| coated synthetic phlogopite *¹ | | | | | | | |
| Aluminum hydroxide-coated | | 6 | 6 | 6 | 6 | 6 | 6 |
| titanium oxide *² | | | | | | | |
| Triethoxycaprylylsilane- | | 0.32 | 0.32 | 0.32 | 0.32 | 0.32 | 0.32 |
| coated red iron oxide *³ | | | | | | | |
| Triethoxycaprylylsilane- | | 0.8 | 0.8 | 0.8 | 0.8 | 0.8 | 0.8 |
| coated yellow iron oxide *⁴ | | | | | | | |
| Triethoxycaprylylsilane- | | 0.001 | 0.001 | 0.001 | 0.001 | 0.001 | 0.001 |
| coated black iron oxide *⁵ | | | | | | | |
| Dipropylene glycol | | 5 | 7 | 12 | 17 | 22 | 25 |
| PEG-240/HDI copolymer bis- | | 0.9 | 0.9 | 0.9 | 0.9 | 0.9 | 0.9 |
| decyltetradeceth-20 ether *⁶ | | | | | | | |
| Ammonium acryloyldimethyltaurate/VP | | 0.3 | 0.3 | 0.3 | 0.3 | 0.3 | 0.3 |
| copolymer *⁷ | | | | | | | |
| Triethylhexanoin | | 5 | 5 | 5 | 5 | 5 | 5 |
| Phenoxyethanol | | 0.5 | 0.5 | 0.5 | 0.5 | 0.5 | 0.5 |
| Ion-exchanged water | | Balance | Balance | Balance | Balance | Balance | Balance |
| Total | | 100 | 100 | 100 | 100 | 100 | 100 |
| Evaluation | Dispersibility | E | A | A | A | A | B |
| | Moistness | B | B | B | B | B | B |
| | Lack of squeakiness | D | C | B | B | A | A |
| | Uniformity of coating film | C | B | A | B | C | C |
| | Lack of stickiness | A | A | A | B | C | C |
| | Luster in application region | D | C | B | B | B | B |

*¹ Platy powder; aspect ratio: 40
*² Blocky powder
*³ Nonspherical powder
*⁴ Nonspherical powder
*⁵ Nonspherical powder
*⁶ Adeka Nol GT700 (from ADEKA Corporation)
*⁷ Aristflex AVC (from Clariant)

### Test Examples 7 to 9

Powder-containing compositions were prepared by changing the types of liquid-state polyols. All the liquid-state polyols were able to obtain good ratings. Particularly, by using dihydric alcohols, it was possible to further reduce stickiness.

TABLE 2

| | | Test Example | | |
|---|---|---|---|---|
| | | 7 | 8 | 9 |
| Distearyldimonium chloride-coated synthetic | | 2.879 | 2.879 | 2.879 |
| phlogopite *¹ | | | | |
| Aluminum hydroxide-coated titanium | | 6 | 6 | 6 |
| oxide *² | | | | |
| Triethoxycaprylylsilane-coated red iron | | 0.32 | 0.32 | 0.32 |
| oxide *³ | | | | |
| Triethoxycaprylylsilane-coated yellow iron | | 0.8 | 0.8 | 0.8 |
| oxide *⁴ | | | | |
| Triethoxycaprylylsilane-coated black iron | | 0.001 | 0.001 | 0.001 |
| oxide *⁵ | | | | |
| Dipropylene glycol | | 12 | — | — |
| 1,3-Butylene glycol | | — | 12 | — |
| Glycerin | | — | — | 12 |
| PEG-240/HDI copolymer bis- | | 0.9 | 0.9 | 0.9 |
| decyltetradeceth-20 ether *⁶ | | | | |
| Ammonium acryloyldimethyltaurate/VP | | 0.3 | 0.3 | 0.3 |
| copolymer *⁷ | | | | |
| Triethylhexanoin | | 5 | 5 | 5 |
| Phenoxyethanol | | 0.5 | 0.5 | 0.5 |
| Ion-exchanged water | | Balance | Balance | Balance |
| Total | | 100 | 100 | 100 |
| Evaluation | Dispersibility | A | A | A |
| | Moistness | B | B | B |
| | Lack of squeakiness | B | B | B |
| | Uniformity of coating film | A | A | A |
| | Lack of stickiness | A | A | B |
| | Luster in application region | B | B | B |

### Test Examples 10 to 13

Powder-containing compositions were prepared by changing the content by percentage of the platy powder. Dispersibility deteriorated in Test Example 13 containing 25% by mass of platy powder. Also, the ratings regarding the lack of squeakiness and uniformity of coating film deteriorated. From the above, it is thought that the content of the platy powder is preferably 22% by mass or less, more preferably 18% by mass or less. A good rating was obtained also when the content of the platy powder was 1% by mass.

TABLE 3

| | | Test Example | | | |
|---|---|---|---|---|---|
| | | 10 | 11 | 12 | 13 |
| Distearyldimonium chloride-coated | | 1 | 10 | 20 | 25 |
| synthetic phlogopite *¹ | | | | | |
| Dipropylene glycol | | 8 | 8 | 8 | 8 |
| 1,3-Butylene glycol | | 5 | 5 | 5 | 5 |
| Glycerin | | 2 | 2 | 2 | 2 |
| PEG-240/HDI copolymer bis- | | 0.9 | 0.9 | 0.9 | 0.9 |
| decyltetradeceth-20 ether *⁶ | | | | | |
| Ammonium acryloyldimethyltaurate/ | | 0.3 | 0.3 | 0.3 | 0.3 |
| VP copolymer *⁷ | | | | | |
| Triethylhexanoin | | 5 | 5 | 5 | 5 |
| Phenoxyethanol | | 0.5 | 0.5 | 0.5 | 0.5 |
| Ion-exchanged water | | Bal- | Bal- | Bal- | Bal- |
| | | ance | ance | ance | ance |
| Total | | 100 | 100 | 100 | 100 |
| Evalua- | Dispersibility | A | A | A | E |
| tion | Moistness | B | B | B | B |
| | Lack of squeakiness | B | B | C | D |
| | Uniformity of coating film | B | B | C | D |
| | Lack of stickiness | A | B | B | C |
| | Luster in application region | C | B | A | A |

### Test Examples 14 to 16

Powder-containing compositions were prepared by changing the blending ratio between platy particles having an aspect ratio of 40 (the first powder) and spherical particles having an aspect ratio of 1 (the second powder). In Test Example 16, which included only spherical particles and no platy particles, many items had poor ratings. Also in Test Example 15, which included a greater amount of spherical particles than platy powder, the ratings regarding the uniformity of coating film and luster in the application region were poor. From the above, it is thought that the content of the second powder, having an aspect ratio or less than 1.2, to 1 part by mass of the first powder, having an aspect ratio of 1.5 or greater, is preferably 1.5 parts by mass or less.

TABLE 4

| | | Test Example | | |
|---|---|---|---|---|
| | | 14 | 15 | 16 |
| Distearyldimonium chloride-coated synthetic | | 15 | 5 | 0 |
| phlogopite *¹ | | | | |
| Methyl methacrylate crosspolymer *⁸ | | 0 | 10 | 15 |
| Triethoxycaprylylsilane-coated red iron | | 0.32 | 0.32 | 0.32 |
| oxide *³ | | | | |
| Triethoxycaprylylsilane-coated yellow iron | | 0.8 | 0.8 | 0.8 |
| oxide *⁴ | | | | |
| Triethoxycaprylylsilane-coated black iron | | 0.001 | 0.001 | 0.001 |
| oxide *⁵ | | | | |
| Dipropylene glycol | | 8 | 8 | 8 |
| 1,3-Butylene glycol | | 5 | 5 | 5 |
| Glycerin | | 2 | 2 | 2 |
| PEG-240/HDI copolymer bis- | | 0.9 | 0.9 | 0.9 |
| decyltetradeceth-20 ether *⁶ | | | | |
| Ammonium acryloyldimethyltaurate/VP | | 0.3 | 0.3 | 0.3 |
| copolymer *⁷ | | | | |
| Triethylhexanoin | | 5 | 5 | 5 |
| Phenoxyethanol | | 0.5 | 0.5 | 0.5 |
| Ion-exchanged water | | Balance | Balance | Balance |
| Total | | 100 | 100 | 100 |
| Evalua- | Dispersibility | A | A | A |
| tion | Moistness | B | B | B |
| | Lack of squeakiness | B | B | E |
| | Uniformity of coating film | B | D | E |
| | Lack of stickiness | B | A | A |
| | Luster in application region | B | D | E |

*⁸ Spherical powder; aspect ratio: 1

### Test Examples 17 to 26

Powder-containing compositions were prepared by preparing platy powders and a blocky powder by changing the hydrophobizing methods. All the Test Examples were able to obtain good ratings. Particularly, powders treated by magnesium stearate and distearyldimonium chloride were able to obtain even better ratings.

Test Example 26, which did not include a platy powder but included a blocky powder, was also able to obtain good ratings.

TABLE 5

| Test Example | | 17 | 18 | 19 | 20 | 21 |
|---|---|---|---|---|---|---|
| Magnesium stearate-coated | | 3 | — | — | — | — |
| synthetic phlogopite *⁹ | | | | | | |
| Triethoxysilylethyl | | — | 3 | — | — | — |
| polydimethylsiloxyethyl | | | | | | |
| dimethicone-coated synthetic | | | | | | |
| phlogopite *¹⁰ | | | | | | |
| Distearyldimonium chloride- | | — | — | 3 | — | — |
| coated synthetic phlogopite *¹¹ | | | | | | |
| Synthetic phlogopite *¹² | | — | — | — | 3 | — |
| Distearyldimonium chloride- | | — | — | — | — | 3 |
| coated muscovite *¹³ | | | | | | |
| Aluminum hydroxide-coated | | 6 | 6 | 6 | 6 | 6 |
| titanium oxide *² | | | | | | |
| Triethoxycaprylylsilane-coated | | 0.32 | 0.32 | 0.32 | 0.32 | 0.32 |
| red iron oxide *³ | | | | | | |
| Triethoxycaprylylsilane-coated | | 0.8 | 0.8 | 0.8 | 0.8 | 0.8 |
| yellow iron oxide *⁴ | | | | | | |
| Triethoxycaprylylsilane-coated | | 0.001 | 0.001 | 0.001 | 0.001 | 0.001 |
| black iron oxide *⁵ | | | | | | |
| Dipropylene glycol | | 12 | 12 | 12 | 12 | 12 |
| PEG-240/HDI copolymer bis- | | 0.9 | 0.9 | 0.9 | 0.9 | 0.9 |
| decyltetradeceth-20 ether *⁶ | | | | | | |
| Ammonium acryloyldimethyltaurate/VP | | 0.3 | 0.3 | 0.3 | 0.3 | 0.3 |
| copolymer *⁷ | | | | | | |
| Triethylhexanoin | | 5 | 5 | 5 | 5 | 5 |
| Phenoxyethanol | | 0.5 | 0.5 | 0.5 | 0.5 | 0.5 |
| Ion-exchanged water | | Balance | Balance | Balance | Balance | Balance |
| Total | | 100 | 100 | 100 | 100 | 100 |
| Evaluation | Dispersibility | A | A | A | A | A |
| | Moistness | B | B | B | B | B |
| | Lack of squeakiness | A | B | B | A | A |
| | Uniformity of coating film | A | B | A | B | A |
| | Lack of stickiness | A | A | A | A | A |
| | Luster in application region | B | B | B | B | B |

*⁹ Platy powder; aspect ratio: 20
*¹⁰ Platy powder; aspect ratio: 20
*¹¹ Platy powder; aspect ratio: 20
*¹² Platy powder; aspect ratio: 20
*¹³ Platy powder; aspect ratio: 20

TABLE 6

| Test Example | | 22 | 23 | 24 | 25 | 26 |
|---|---|---|---|---|---|---|
| Distearyldimonium chloride- | | 3 | — | — | — | — |
| coated sericite *¹⁴ | | | | | | |
| Amodimethicone-coated mica *¹⁵ | | — | 3 | — | — | — |
| Hydrogen dimethicone-coated | | — | — | 3 | — | — |
| synthetic phlogopite iron *¹⁶ | | | | | | |
| Hydrogen dimethicone-coated | | — | — | — | 3 | — |
| calcined sericite *¹⁷ | | | | | | |
| Zinc decyl trisiloxanecarboxylate- | | | | | | 3 |
| treated talc *¹⁸ | | | | | | |
| Aluminum hydroxide-coated | | 6 | 6 | 6 | 6 | 6 |
| titanium oxide *² | | | | | | |
| Triethoxycaprylylsilane-coated | | 0.32 | 0.32 | 0.32 | 0.32 | 0.32 |
| red iron oxide *³ | | | | | | |
| Triethoxycaprylylsilane-coated | | 0.8 | 0.8 | 0.8 | 0.8 | 0.8 |
| yellow iron oxide *⁴ | | | | | | |
| Triethoxycaprylylsilane-coated | | 0.001 | 0.001 | 0.001 | 0.001 | 0.001 |
| black iron oxide *⁵ | | | | | | |
| Dipropylene glycol | | 12 | 12 | 12 | 12 | 12 |
| PEG-240/HDI copolymer bis- | | 0.9 | 0.9 | 0.9 | 0.9 | 0.9 |
| decyltetradeceth-20 ether *⁶ | | | | | | |
| Ammonium acryloyldimethyltaurate/VP | | 0.3 | 0.3 | 0.3 | 0.3 | 0.3 |
| copolymer *⁷ | | | | | | |
| Triethylhexanoin | | 5 | 5 | 5 | 5 | 5 |
| Phenoxyethanol | | 0.5 | 0.5 | 0.5 | 0.5 | 0.5 |
| Ion-exchanged water | | Balance | Balance | Balance | Balance | Balance |
| Total | | 100 | 100 | 100 | 100 | 100 |
| Evaluation | Dispersibility | A | A | A | A | A |
| | Moistness | B | B | B | B | B |
| | Lack of squeakiness | A | B | B | B | B |
| | Uniformity of coating film | A | B | B | B | A |
| | Lack of stickiness | A | A | A | A | A |
| | Luster in application region | B | B | B | B | B |

*¹⁴ Platy powder; aspect ratio: 20
*¹⁵ Platy powder; aspect ratio: 20
*¹⁶ Platy powder; aspect ratio: 20
*¹⁷ Platy powder; aspect ratio: 20
*¹⁸ Blocky powder; aspect ratio: 3 to 5

### Test Examples 27 to 31

Powder-containing compositions were prepared by changing the content by percentage of the liquid-state oily component. In Test Example 27, which included 1% by mass of the liquid-state oily component, the rating regarding moistness was poor. From the above, it is thought that the content of the liquid-state oily component relative to the mass of the powder-containing composition is preferably 3% by mass or greater, more preferably 5% by mass or greater. In Test Example 31, which included 15% by mass of the liquid-state oily component, the ratings regarding dispersibility and lack of stickiness were poor. From the above, it is thought that the content of the liquid-state oily component relative to the mass of the powder-containing composition is preferably 12% by mass or less, more preferably 10% by mass or less.

TABLE 7

| Test Example | | 27 | 28 | 29 | 30 | 31 |
|---|---|---|---|---|---|---|
| Distearyldimonium chloride- | | 2.879 | 2.879 | 2.879 | 2.879 | 2.879 |
| coated synthetic phlogopite *¹ | | | | | | |
| Aluminum hydroxide-coated | | 6 | 6 | 6 | 6 | 6 |
| titanium oxide *² | | | | | | |
| Triethoxycaprylylsilane-coated | | 0.32 | 0.32 | 0.32 | 0.32 | 0.32 |
| red iron oxide *³ | | | | | | |
| Triethoxycaprylylsilane-coated | | 0.8 | 0.8 | 0.8 | 0.8 | 0.8 |
| yellow iron oxide *⁴ | | | | | | |
| Triethoxycaprylylsilane-coated | | 0.001 | 0.001 | 0.001 | 0.001 | 0.001 |
| black iron oxide *⁵ | | | | | | |
| Dipropylene glycol | | 8 | 8 | 8 | 8 | 8 |
| 1,3-Butylene glycol | | 5 | 5 | 5 | 5 | 5 |
| Glycerin | | 2 | 2 | 2 | 2 | 2 |
| PEG-240/HDI copolymer bis- | | 0.7 | 0.7 | 0.7 | 0.7 | 0.7 |
| decyltetradeceth-20 ether *⁶ | | | | | | |
| Ammonium acryloyldimethyltaurate/VP | | 0.3 | 0.3 | 0.3 | 0.3 | 0.3 |
| copolymer *⁷ | | | | | | |
| Triethylhexanoin | | 1 | 5 | 7 | 10 | 15 |
| Phenoxyethanol | | 0.5 | 0.5 | 0.5 | 0.5 | 0.5 |
| Ion-exchanged water | | Balance | Balance | Balance | Balance | Balance |
| Total | | 100 | 100 | 100 | 100 | 100 |
| Evaluation | Dispersibility | D | B | A | A | A |
| | Moistness | A | A | A | A | E |
| | Lack of squeakiness | A | A | A | A | A |
| | Uniformity of coating film | A | A | A | B | C |
| | Lack of stickiness | A | B | B | C | D |
| | Luster in application region | A | A | A | B | C |

The powder-containing composition according to the present invention have been described according to the foregoing embodiments and examples, but the invention is not limited to the foregoing embodiments and examples and may encompass various transformations, modifications, and improvements made to the various disclosed elements (including elements disclosed in the Claims, Description, and Drawings) within the scope of the invention and according to the fundamental technical idea of the present invention. Further, various combinations, substitutions, and selections of the various disclosed elements are possible within the scope of the claims of the invention.

Further issues, objectives, and embodiments (including modifications) of the present invention are revealed also from the entire disclosure of the invention including the Claims.

The numerical ranges disclosed herein are to be construed in such a manner that arbitrary numerical values and ranges falling within the disclosed ranges are treated as being concretely described herein, even where not specifically stated.

### INDUSTRIAL APPLICABILITY

The powder-containing composition of the present disclosure is applicable, for example, to external-use skin preparations, cosmetics, cleansers, etc., applicable to the skin. The powder-containing composition of the present disclosure is applicable, for example, to base makeup, veil makeup, makeup cosmetics, antiperspirants, deodorants, sun-block cosmetics, skin-care agents, cleansers, etc.

Some or all of the foregoing embodiments may be described as in the following additional features, although not limited thereto. The various additional features may be employed in combination with the claim(s) in the Scope of Claims.

### ADDITIONAL FEATURE 1

A method of using a powder-containing composition, comprising using the powder-containing composition of the present disclosure as an external-use skin preparation.

### ADDITIONAL FEATURE 2

A method of using a powder-containing composition, comprising using the powder-containing composition of the present disclosure as a cosmetic.

The powder-containing composition of the present disclosure is applicable to coating agents applicable to objects other than the skin. For example, the powder-containing composition of the present disclosure is applicable to paint materials, coating agents, etc.

1. A powder-containing composition comprising:
from 0.5% by mass to 22% by mass of a first powder which is a nonspherical powder having a hydrophobic particle surface;
from 6% by mass to 25% by mass of a liquid-state polyol;
50% by mass or greater of water; and
from 3% by mass to 12% by mass of a liquid-state oily component, wherein:
a content of a second powder which is a spherical powder is 1.5 parts by mass or less relative to 1 part by mass of the first powder; and
a content of a surfactant is 0.5% by mass or less relative to the mass of the powder-containing composition.

2. The powder-containing composition according to claim 1, wherein the first powder is at least one selected from the group consisting of platy powders and blocky powders.

3. The powder-containing composition according to claim 1, wherein the first powder has an aspect ratio of from 1.5 to 170.

4. The powder-containing composition according to claim 1, wherein the second powder has an average aspect ratio of less than 1.2.

5. The powder-containing composition according to claim 1, wherein the oily component is at least one selected from the group consisting of fluoro-oils, silicone oils, polar oils, and hydrocarbon oils.

6. The powder-containing composition according to claim 1, wherein the first powder is a platy powder coated by at least one selected from the group consisting of higher fatty acids, metallic soaps, silicone compounds, surfactants, and dextrin fatty acid esters.

7. The powder-containing composition according to claim 1, wherein the liquid-state polyol is at least one selected from the group consisting of glycerin, 1,3-butylene glycol, polyethylene glycol, diglycerin, and dipropylene glycol.

8. The powder-containing composition according to claim 1, further comprising at least one selected from the group consisting of PEG-240/HDI copolymer bis-decyltetradeceth-20 ether, dimethylacrylamide/sodium acryloyldimethyltaurate crosspolymer, and polyurethane-59.

9. The powder-containing composition according to claim 1, wherein the powder-containing composition has a viscosity of from 1,000 mPa·s to 150,000 mPa·s.

10. The composition according to claim 1, wherein the liquid-state oily component and the water are a mixture in a state of a giant emulsion.

11. The powder-containing composition according to claim 1, wherein the composition is for use in a cosmetic.

12. The powder-containing composition according to claim 2, wherein the first powder has an aspect ratio of from 1.5 to 170.

13. The powder-containing composition according to claim 3, wherein the second powder has an average aspect ratio of less than 1.2.

14. The powder-containing composition according to claim 5, wherein the first powder is a platy powder coated by at least one selected from the group consisting of higher fatty acids, metallic soaps, silicone compounds, surfactants, and dextrin fatty acid esters.

15. The powder-containing composition according to claim 5, wherein the liquid-state polyol is at least one selected from the group consisting of glycerin, 1,3-butylene glycol, polyethylene glycol, diglycerin, and dipropylene glycol.

16. The powder-containing composition according to claim 6, wherein the liquid-state polyol is at least one selected from the group consisting of glycerin, 1,3-butylene glycol, polyethylene glycol, diglycerin, and dipropylene glycol.

17. The powder-containing composition according to claim 5, further comprising at least one selected from the group consisting of PEG-240/HDI copolymer bis-decyltetradeceth-20 ether, dimethylacrylamide/sodium acryloyldimethyltaurate crosspolymer, and polyurethane-59.

18. The powder-containing composition according to claim 6, further comprising at least one selected from the group consisting of PEG-240/HDI copolymer bis-decyltetradeceth-20 ether, dimethylacrylamide/sodium acryloyldimethyltaurate crosspolymer, and polyurethane-59.

19. The powder-containing composition according to claim 7, further comprising at least one selected from the group consisting of PEG-240/HDI copolymer bis-decyltetradeceth-20 ether, dimethylacrylamide/sodium acryloyldimethyltaurate crosspolymer, and polyurethane-59.

20. The powder-containing composition according to claim 16, further comprising at least one selected from the group consisting of PEG-240/HDI copolymer bis-decyltetradeceth-20 ether, dimethylacrylamide/sodium acryloyldimethyltaurate crosspolymer, and polyurethane-59.
